(19) United States
(12) Patent Application Publication
(10) Pub. No.: US 2010/0160772 A1
(43) Pub. Date: Jun. 24, 2010

(54) Adaptable Image Guided Delivery System

(75) Inventors: Kenneth C. Gardeski, Plymouth, MN (US); Sonar Shah, Los Angeles, CA (US); Michael R. Neidert, Salthill (IE)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/630,325

(22) Filed: Dec. 3, 2009

Related U.S. Application Data
(60) Provisional application No. 61/138,738, filed on Dec. 18, 2008.

(51) Int. Cl. A61B 5/05

(52) U.S. Cl. 600/424

(57) ABSTRACT

A navigation element for delivery of a therapy delivery system and method of enabling navigation of a therapy delivery system. The navigation element for a therapy delivery system comprises a flexible elongate tubular sheath having a lumen extending longitudinally therethrough, the lumen of the tubular body being sized to fit over a catheter; and an electromagnetic receiver assembly within the tubular body, the receiver assembly comprising a receiver coil and a conductor coupled to the receiver coil, the conductor coupled to the receiver coil and extending towards a proximal end of the tubular sheath.

### CROSS-REFERENCE TO RELATED APPLICATIONS

This application claims the benefit of U.S. Provisional Application Ser. No. 61/138,738 filed Dec. 18, 2008, entitled “Adaptable Image Guided Delivery System”, the contents of which are incorporated by reference herein in its entirety.

### BACKGROUND

Certain medical devices access the inside of the body through minimally invasive techniques. For example, cardiac catheters, ablation catheters and leads access the heart by threading through the vascular system. Physicians who perform procedures using these devices rely in part upon experience and the known anatomy for appropriate placement. However, the accuracy and ease of placement can be greatly enhanced by using image guidance for the device.

Certain devices are designed to use an electromagnetic source and electromagnetic detectors or receivers for minimally invasive surgical implantation procedures. The source, positioned external to the patient, sets up a magnetic field that induces a voltage in receivers mounted on the surgical instrument or delivery tool, which has been inserted within the body of the patient disposed within the magnetic field. The voltage of each receiver is dependent upon the location and orientation of the respective receiver within the magnetic field. By sensing and processing current conducted from each receiver, a navigation analysis system can determine the location of each receiver with respect to one another and provide a visual map to aid the operator in navigating the device to a target site within the body of the patient. Such systems provide the advantages of imaging with reduced radiation exposure and provide three-dimensional imaging.

Some devices which are used in minimally invasive surgical implantation procedures lack a navigation system. Rather than redesign the device, it is desirable to provide a way to retrofit such devices with navigation capabilities when such capabilities are desired. One way of retrofitting a medical device for use with a navigation system is disclosed in U.S. patent application Publication Number 2007/0164900. However, the disclosed navigation element requires that the medical device have a sufficiently large lumen in order to accommodate the navigation element. Certain medical devices, such as those which lack lumens or have small lumens, cannot be used with this navigation element. It is therefore desirable to provide an alternative device for retrofitting existing medical devices with navigation systems.

### SUMMARY

In one embodiment, the navigation element comprises a flexible elongate tubular sheath having a lumen extending longitudinally through the elongate sheath. The lumen of the tubular body is sized and is adapted to fit over a catheter. The navigation element further comprises an electromagnetic receiver assembly within the tubular body, wherein the receiver assembly comprises a receiver coil and a conductor coupled to the receiver coil, the conductor coupled to the receiver coil and extending towards a proximal end of the tubular sheath.

In another embodiment, the navigation element comprises an inner tubular body having an interior surface and an exterior surface and having a lumen extending longitudinally through the elongate sheath. The lumen of the tubular body is sized and is adapted to fit over an elongate medical device. The navigation element further comprises an electromagnetic receiver assembly comprising a receiver coil wound around a portion of the exterior surface of the inner tubular body and an outer tubular body having a an interior surface and an exterior surface and having a lumen extending longitudinally therethrough, the interior surface of the outer tubular body being adjoined to the exterior surface of the inner tubular body and the electromagnetic receiver assembly.

### BRIEF DESCRIPTION OF THE DRAWINGS

The following drawings are illustrative of particular embodiments of the present invention and therefore do not limit the scope of the invention. The drawings are intended for use in conjunction with the explanations in the following detailed description. Embodiments of the present invention will hereinafter be described in conjunction with the appended drawings, wherein like numerals denote like elements.

FIG. 1 is a schematic view of an exemplary navigation element;

FIG. 2 is a perspective view of a distal portion of a navigation element;

FIG. 3 is a perspective view of a distal portion of a navigation element;

FIG. 4 is an electromagnetic receiver assembly according;

FIG. 5 is cross-section of a navigation element;

FIG. 6 is a virtual fluoroscopy image of a navigation element;

FIG. 7 is a partially assembled navigation element; and

FIG. 8 is a navigation element surrounding a delivery catheter and lead.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

Exemplary navigation elements provide navigation capability to medical devices which lack navigation capability. This navigation capability allows the clinician to identify the location of the navigation element, and therefore of the medical device, within the body. While such medical device could each be redesigned to include navigation, embodiments presented herein make such redesign unnecessary. Rather, they allow these medical devices to be retrofit with navigation elements, eliminating the need for redesign of each medical device and allowing a single medical device to be used optionally with or without navigation.

One embodiment of the navigation element is designed as a sheath which fits over the medical device. As such, the navigation element is preferably flexible so that it does not interfere with the normal functioning of the medical device. The navigation element may therefore be steered or advanced into position by the normal mechanisms of the medical device such that the navigation element is passively advanced along with the medical device. In addition, the navigation element is preferably thin, so that it only minimally increases the profile of the medical device. In this way, the navigation does not interfere with the vascular access or other types of access through which the medical device is typically used. The choice of material for the navigation element should consider the need for flexibility and thinness. For example, the navigation element may be made from a short length of copper. Alternatively, the navigation element may be made from a polymer with a flexural moduli below 25,000 psi and preferably below 5,000 psi. Useful polymers that satisfy the flexural moduli range include silicone, polyurethane such as DOW PELLETHANE® 2363-80A polyurethane, ARKEMA PEBAX® 3533 SN-00 polyether block amide, or DSM Arnitel EL630 polyetherester or a combination thereof.

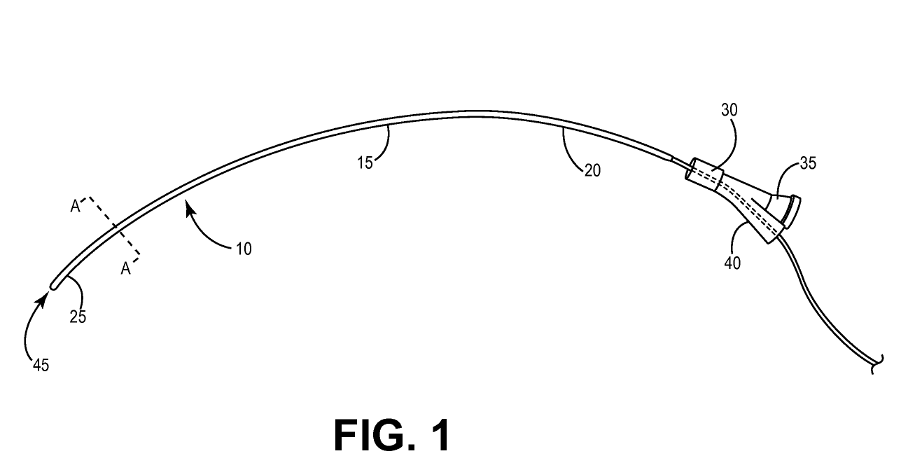

An example of a navigation element 10 is shown in FIG. 1. The navigation element 10 comprises an elongated tubular body 15 having a proximal end 20 and a distal end 25. The proximal end 20 includes a hub 30 that branches off into a locking mechanism 35 and an off-line attachment 40, as further described below.

The navigation element 10 has lumen 45 that extends longitudinally through the elongate tubular body 15 from the proximal end 20 to the distal end 25. The lumen 45 is sized to allow insertion of a medical device into the lumen. However, such medical devices may be available in a variety of sizes. In order to accommodate various medical devices having a variety of sized, navigation elements 10 may be provided having lumens of various sizes and various lengths and relatively sized to accommodate a particular class of device (e.g., 2 French, 4 French, 6 French, etc.).

A variety of medical devices may be used with the navigation element of the invention. However, such medical devices generally have an elongated shape to allow them to be inserted into the navigation element. Examples of appropriate medical devices include therapy delivery devices such as catheters. Such catheters may be used for lead delivery, ablation or diagnostic purposes, for example. Other medical devices which may be used with embodiments of the invention include electrophysiology catheters. For some procedures, electrophysiology catheters may not need navigation. However, for other procedures, such as more complex or difficult procedures, navigation may be useful for providing more certainty regarding the location of the electrophysiology catheter. As such, embodiments of the invention are particularly useful in that they provide the clinician with the option of using the same electrophysiology catheter either with or without the navigation element, depending upon the particular procedure and the clinician's preference.

Other appropriate medical devices include endoscopy devices. For example, devices such as endoscopes for use in gastroenterology (e.g. colonoscopy, rectosigmoidoscopy, lanryngoscopy), pulmonology (e.g. pulmonary biopsies), orthopaedics and neurology (e.g. neuroendoscopy for placement of hydrocephalus shunts). While endoscopes provide internal visualization, the precise location of the endoscope within the body may not always be clear to the physician throughout the procedure, making a navigation attachment useful, particularly during delicate or complex procedures. For example, the navigation element may be placed over a neuroendoscope to provide navigation during neurological procedures, including cranial applications such as the placement of a shunt within the brain.

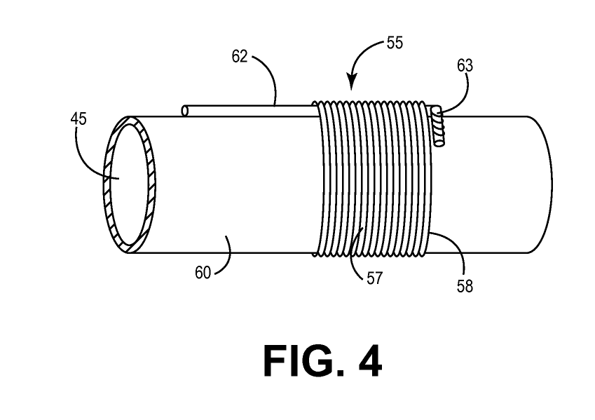

In order to provide navigation functionality, the navigation element 10 includes one or more electromagnetic receiver assemblies 55 (FIG. 4). The electromagnetic receiver assembly 55 is located within the elongate tubular body 15 such that it is isolated and protected from blood or other fluids or tissue when the navigation element is being used within a patient. In some embodiments, the electromagnetic receiver assembly 55 is comprised of a coiled wire 57 and a conductor 62 coupled to the coiled wire. In some embodiments, the coiled wire 57 is within the elongate tubular body 15 and forms one or more loops around the lumen 45 of the elongate tubular body 15. By looping around the lumen 45, the coiled wire occupies a minimum volume within the elongate tubular 15 body so that the navigation element remains as thin as possible.

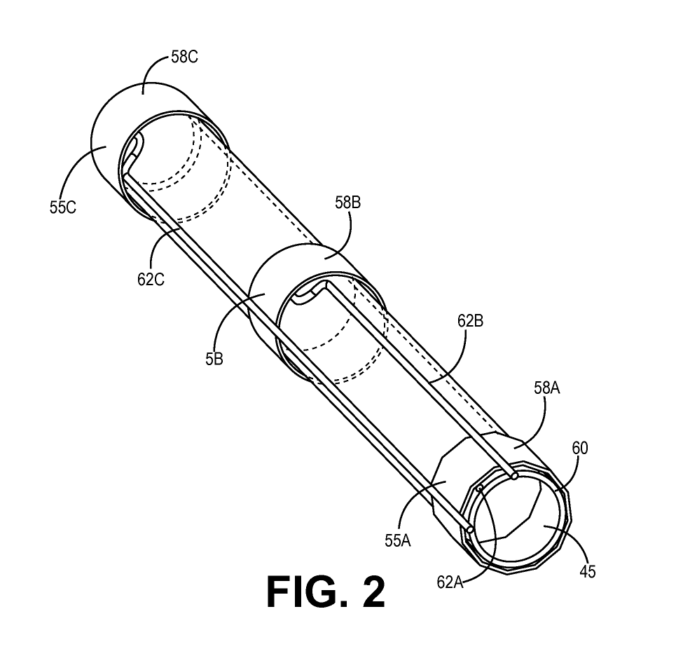

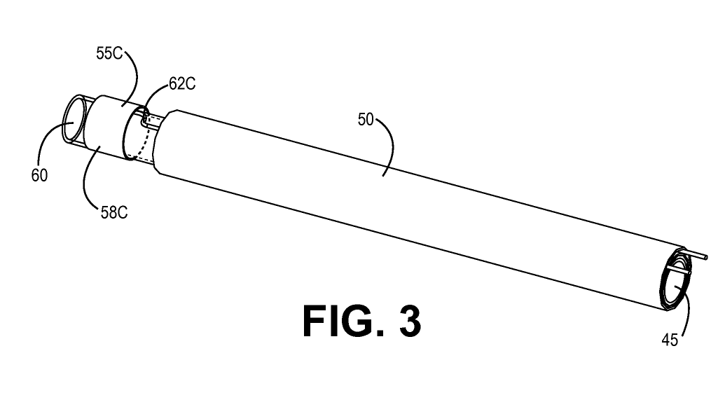

Embodiments of the navigation element 10 comprise an outer tubular body 50 and an inner tubular body 60. FIG. 2 illustrates a portion of the elongate body 15 of the navigation element 10 with the outer tubular body 50 not shown, to provide a view of the components within the outer tubular body 50, while FIG. 3 shows a portion of the elongate body including the outer tubular body 50. The outer tubular body 50 extends from hub 30 to distal end 25 over receiver assemblies 55A, 55B, 55C, and the inner tubular body 60 extends within the lumen of outer tubular body 50 along a length of outer tubular body 50 and within receiver assemblies 55A, 55B, 55C. The distal ends of the inner and outer tubular bodies 60, 50 are coupled together such that the lumen 45 of the inner tubular body 60, extends to the distal end 25. Lumen 45 may be used to closely receive and/or fit over a medical device, such as a catheter.

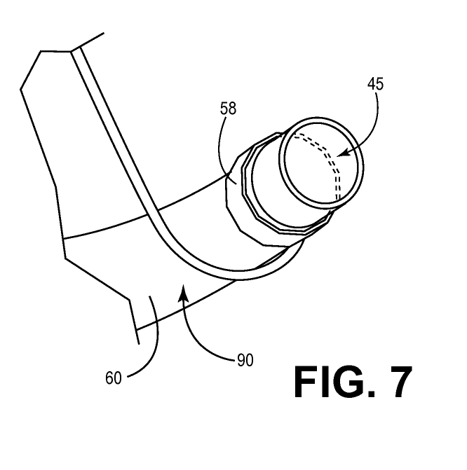

In some embodiments such as that shown in FIGS. 2 and 3, the navigation element 10 includes multiple electromagnetic receiver assemblies, such as three electromagnetic receiver assemblies 55A, 55B, 55C. The electromagnetic receiver assemblies 55A, 55B, 55C are located at the distal end 25 of the elongated tubular body 15 and may be spaced apart from each other, such as 50 millimeters. However, this distance may be greater or lesser, depending upon the intended use of the medical device or the intended location of insertion, for example. For example, when the navigation element is to be used in a more tortuous anatomic location, a navigation element having closer spacing between the coils may be provided. FIG. 2 further illustrates each receiver assembly 55A, 55B, 55C including a respective receiver coil 58A, 58B, 58C, and conductors 62A, 62B and 62C extending proximally therefrom, within the lumen 105 of outer tubular body 50, and outside inner tubular body 60 and proximal receiver coils. For example, conductor 62C extends outside of coils 58B, 58C and conductor 62B extends outside of coil 58A; conductors 62A, 62B and 62C further extend into a lumen of hub 30 and then into an off-line attachment 40 of hub 30. According to the some embodiments such as the embodiment shown in FIG. 7, off-line attachment extends laterally from the hub lumen to route conductors 62A, 62B and 62C into insulated extension 65 for coupling with a connector, and inner tubular body 60 extends within hub 30 proximally such that the lumen 45 is in proximity to hub entry 75. Hub entry 75 is designed to facilitate introduction of the aforementioned catheter into its lumen. Hub entry 75 is further shown including a valve 80 that may be used for coupling to and fixing the catheter in place relative to the navigation element 10. Connector 70 is connectable to an analysis component of the navigation system (not shown).

FIG. 4 depicts one of the receiver assemblies 55 (e.g., 55A, 55B, 55C) for a navigation element 10, shown in FIG. 1. The receiver assembly 55 shown in FIG. 4 includes a coiled wire wound in loops to form a receiver coil 58, and a conductor 62 coupled to the coil 58. The coiled wire forms loops around the inner tubular body 60. In the embodiment shown in FIG. 4, the conductor is stripped of insulation and split. The coil wire 57 is wrapped around a single stripped and split twisted pair conductor 63 and the conductor 63 and coil wire 57 are soldered together.

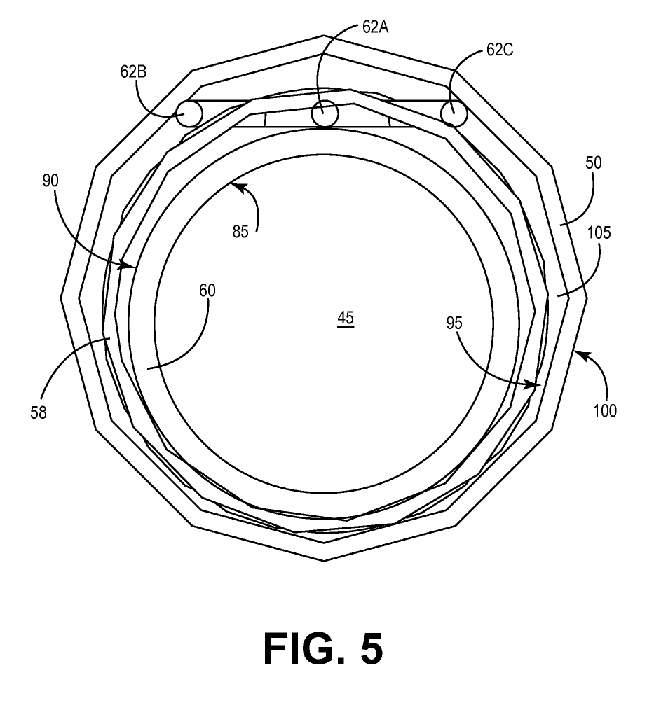

FIG. 5 is a cross-section of the navigation element of FIG. 1 through line A-A. As shown in FIGS. 4 and 5, the elongate tubular body 15 is comprised of an inner tubular body 60 and an outer tubular body 50. The inner tubular body 60 has an interior surface 85 and an exterior surface 90 and has a lumen 45 (the same as lumen 45 defined above) extending longitudinally though the length of the inner tubular body 60. The lumen 45 is sized to fit over a particular elongate medical device or a variety of elongate medical devices. The outer tubular body 50 has an interior surface 95 and an exterior surface 100 and has a lumen 105 extending through the length of it. The interior surface 95 of the outer tubular body 50 is adjoined to the exterior surface 90 of the inner tubular body 60. One or more electromagnetic receiver coils 58 (e.g., 58A, 58B, 58C) are located between the inner tubular body 60 and the outer tubular body 50. In some embodiments, the electromagnetic receiver assembly 55 comprises a wire coil 58 which forms one or more loops around the exterior surface 90 of the inner tubular body 60. The loops are covered by the interior surface 95 of the outer tubular body 50.

According to an exemplary embodiment, wire forming coil 58 has a diameter ranging from approximately 0.00119 inch to approximately 0.00129 inch and is made from a copper alloy, i.e. Electrolytic Tough Pitch (ETP) Copper (CDA alloy no. 11000), having an insulative coating of polyurethane nylon over-coated with a polyvinyl butyral adhesive. The wire may be wound in about six layers of windings, each layer having approximately 80 turns per layer resulting in approximately 480±20 turns for coil 58 that gives receiver assembly 55 an approximate inductance of 140 μH.

Conductor 62 includes first and second insulated wires extending alongside one another that may be formed of a copper alloy, i.e. Electrolytic Tough Pitch (ETP) Copper (CDA alloy no. 11000) having an insulative coating of polyimide and may be twisted about one another along a majority of a length of conductor 62 and held together by an over-jacket of insulation, for example made from a perfluoroalkoxy fluoropolymer resin (PFA). According to an exemplary embodiment, the wires forming conductor 62 have a diameter ranging from approximately 0.0015 inch to approximately 0.004 inch. In some embodiments, the wires are soldered to opposite sides of the receiver coil 58.

The electromagnetic receiver assembly 55 may be used to provide the location of the navigation element 10 and the associated medical device using a navigation analysis system. One navigation analysis system is a system often called virtual fluoroscopy. In virtual fluoroscopy, an analysis component of the navigation analysis system processes current signals from the electromagnetic receiver assemblies. Examples of such systems include the Medtronic StealthStation and the system disclosed in U.S. patent application Publication Number 2004/0097804, the relevant portions of which are hereby incorporated by reference.

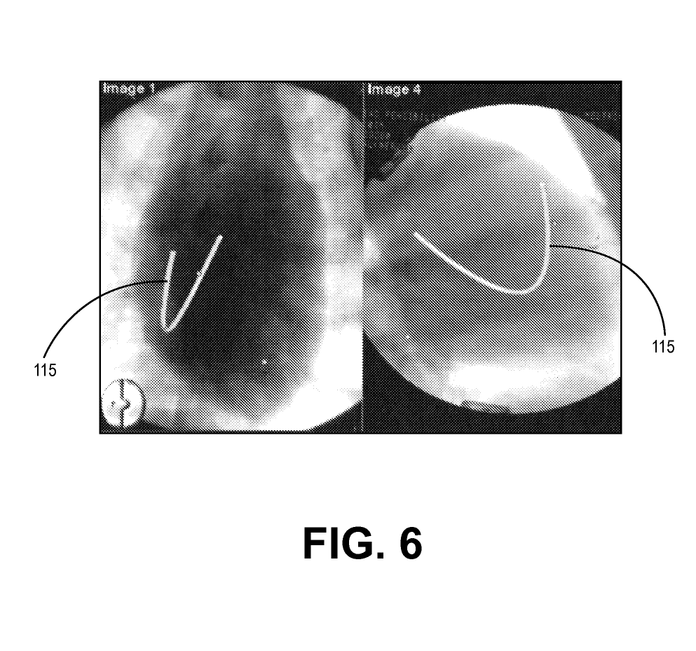

An exemplary virtual fluoroscopy image of a navigation element 10 depicted in FIG. 6. The navigation element 10 was placed over a catheter which lacked visualization capabilities. FIG. 6 provides images 115 of the navigation element 10 in the heart in an anterior-posterior view (left) and a lateral view (right).

The navigation element may be assembled in several operations. An inner elongate tubular sheath 60 which is thin and flexible is provided. The inner elongate tubular body 60 has a lumen 45 which is sized to fit a desired medical device. As shown, for instance in FIG. 7, a wire coil 58 is then wound directly onto and around the outer surface 90 of the inner tubular body 60 at a certain distance from the distal tip of the inner elongate tubular body 60. Alternatively, a bobbin may be placed around the inner elongate tubular member and the wire coil may be wound around the bobbin. Other wire coils 58 are similarly wound around the inner elongate tubular body 60, spaced apart from each other at a distance between them to provide adequate virtual representation of the navigation element. A conductor 62 (FIG. 2) is connected to the wire coil 58, such as by soldering the conductor 62 to the proximal end of the wire coil 58. The conductor 62 is then placed longitudinally along the exterior surface 90 of the inner tubular body 60. The process may be repeated each time another wire coil 58 is added. The conductor 62 associated with each wire coil 58 may extend under all of the more proximate wire coils. Alternatively, the conductor 62 may extend over the more proximate wire coils. In embodiments in which the wire coil is coiled onto a bobbin, the conductor may extend under the bobbin of a more proximate coil or over the more proximal coil. An outer tubular body 50 is then laid over the inner tubular body 60 and the wire coil 58 to isolate the coil 58 and the conductor 62. The inner and outer tubular bodies 60, 50 are merged at their tips.

The navigation element 10 may include a hub 30 with a locking mechanism 35 and an off-line attachment 40 at the proximal end 20 of the elongate tubular body 15 as shown in FIG. 1. The conductors 62 from the receiver coils 58 may be branched off from the lumen 45 via the off-line attachment 40. The off-line attachment 40 is wired to a connector 70 that attaches to the virtual navigation system.

In one embodiment, the locking mechanism 35 is a valve. In some embodiments, the locking mechanism 35 includes a central lumen which is aligned with the central lumen 45 of the elongate tubular body 15. The medical device is inserted through the lumen of the locking mechanism and into the lumen 45 of the elongate tubular body 15. When the medical device is in the desired position within the elongate tubular body 15, the relative position of the medical device is secured or locked within the elongate tubular body 15 using the locking mechanism 35. In this way, the navigation element 10 moves passively and securely with the medical device as the medical device is maneuvered within the patient and the navigation element 10 maintains the same position relative to the medical device throughout the procedure.

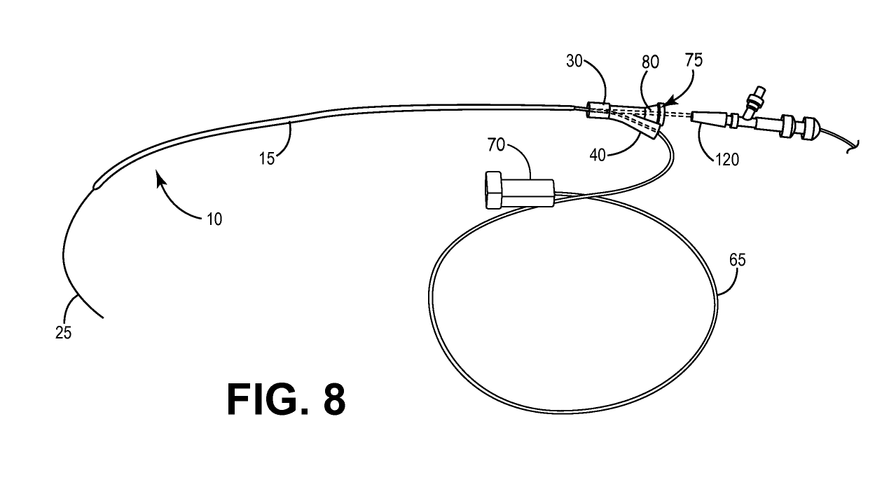

FIG. 8 shows an embodiment of the invention into which a delivery catheter 120 with a 2 French lead 125 has been inserted. The delivery catheter 120 lacks navigation capabilities and the lumen of the delivery catheter 120 is believed to be too small to allow the insertion of a navigation element into the lumen. However, because the navigation element 10 according to embodiments slides over the delivery catheter 120, navigation can be provided for this delivery catheter 120.

Embodiments also include a method of enabling navigation of an elongate medical device. The versatility of the navigation element 10 allows it to be used by a clinician in the field when desired with a variety of medical devices. When the clinician determines that navigation should be used with a particular medical device, a navigation element 10 is selected having a lumen 45 which is sized to fit over the medical device. The medical device is inserted into the lumen 45 of the navigation element 10 the desired distance. In some circumstances, the medical device is advanced within the lumen 45 until the distal end of the medical device is aligned with the distal end 25 of the navigation element 10. In this way, the image 115 of the position of the distal end 25 of the navigation element 10 using the navigation system will correspond with the position of the distal end of the medical device as shown in FIG. 8. However, the medical device may optionally be extended beyond the distal end 25 of the navigation element 10 or may not extend completely to the distal end 25 of the navigation element 10 if the clinician feels such positions are appropriate. When the medical device extends beyond the distal end 25 of the navigation element 10, the distance that the tip of the medical device extends beyond the distal end 25 of the navigation element 10 is input into the navigation system at the beginning of the procedure. Once the medical device is advanced to the desired position within the navigation element 10, it may be secured in position using a locking mechanism 35. In this way, the relative positions of the navigation element 10 and the medical device are maintained while the medical device is positioned within the patient. The medical device with the surrounding navigation element 10 are then inserted into the patient and the medical procedure is performed as usual. The combination of the information about the relative location of the tip of the medical device and the distal end 25 of the navigation element and the position of the electromagnetic receiver assemblies is sufficient to build a virtual representation of the medical device.

In addition to providing navigation capabilities, the navigation element may include sensors in the elongate tubular body. For example, the sensors may be electrical, physiologic or hemodynamic sensors, such as sensors for detecting pressure, oxygen levels, impedance, blood flow, temperature , pH, blood flow, metabolite levels, cardiac motion or dynamic referencing, such as by an accelerometer. In this way, the elongate tubular body can provide additional sensing capabilities to the medical device, as an alternative to, or in addition to, navigational capabilities.

The embodiments and the examples described herein are exemplary and not intended to be limiting in describing the full scope of apparatus, systems, and methods of the present technology. Equivalent changes, modifications and variations of some embodiments, materials, compositions and methods can be made within the scope of the present technology, with substantially similar results.

What is claimed is:

1. A navigation element for a therapy delivery system comprising:
a flexible elongate tubular sheath having a lumen extending longitudinally therethrough, the lumen of the tubular body being sized to fit over a catheter; and
an electromagnetic receiver assembly within the tubular body, the receiver assembly comprising a receiver coil and a conductor coupled to the receiver coil, the conductor coupled to the receiver coil and extending towards a proximal end of the tubular sheath.

2. The navigation element of claim 1, wherein the catheter is an electrophysiology catheter such that the lumen of the tubular body is sized to fit over an electrophysiology catheter.

3. The navigation element of claim 1, wherein the navigation element further comprises a locking mechanism for locking a medical device within the lumen of the navigation element.

4. The navigation element of claim 3, wherein the locking mechanism is located at a proximal end of the tubular body and includes a lumen which is aligned with the lumen of the tubular body.

5. The navigation element of claim 1, wherein the receiver coil comprises a coiled wire.

6. The navigation element of claim 5, wherein the coiled wire forms a coil around the lumen of the tubular body.

7. The navigation element of claim 1, further comprising multiple electromagnetic receiver assemblies within the tubular body.

8. The navigation element of claim 7, wherein the receiver assembly comprises three receiver coils.

9. The navigation element of claim 1 further comprising one or more sensors.

10. A navigation element for a therapy delivery system made comprising:
an inner tubular body having an interior surface and an exterior surface and having a lumen extending longitudinally therethrough, the lumen being sized to fit over an elongate medical device;
an electromagnetic receiver assembly comprising a receiver coil wound around a portion of the exterior surface of the inner tubular body; and
an outer tubular body having a an interior surface and an exterior surface and having a lumen extending longitudinally therethrough, the interior surface of the outer tubular body being adjoined to the exterior surface of the inner tubular body and the electromagnetic receiver assembly.

11. The navigation element of claim 10, wherein the medical device is an electrophysiology catheter.

12. The navigation element of claim 10, further comprising a locking mechanism for locking the medical device within the lumen of the navigation element.

13. The navigation element of claim 12, wherein the locking mechanism is located at a proximal ends of the inner and outer elongate tubular bodies and wherein the locking mechanism includes a lumen which is aligned with the lumen of the inner first elongate tubular body.

14. The navigation element of claim 10, further comprising multiple electromagnetic receiver assemblies within the tubular body.

15. The navigation element of claim 14, wherein the navigation element comprises nine receiver assemblies.

16. A method of enabling navigation of a catheter comprising:
providing a navigation element, the navigation element including a flexible elongate tubular sheath having a lumen extending longitudinally therethrough and having an electromagnetic receiver assembly within the tubular body, the electromagnetic receiver assembly including a receiver coil positioned proximate to a distal end of the navigation element; and
inserting the catheter into the lumen of the navigation element such that the receiver coil is positioned in proximity to a distal end of the catheter.

17. The method of claim 16, wherein at least a portion of the catheter has a solid cross section.

18. The method of claim 16, wherein the navigation element further comprises a locking mechanism.

19. The method of claim 18, further comprising locking the medical device within the navigation element.

20. The method of claim 15, wherein the medical device comprises an electrophysiology catheter.
